(19) United States
(12) Patent Application Publication
(10) Pub. No.: US 2013/0344583 A1
(43) Pub. Date: Dec. 26, 2013

(54) BIOSENSOR COMPRISING AN OXIDASE ENZYME AND A HYDROGEN PEROXIDE INDICATOR MEANS

(71) Applicants: Paul Davis, Felmersham (GB); Andrew Austin, Great Addington (GB)

(72) Inventors: Paul Davis, Felmersham (GB); Andrew Austin, Great Addington (GB)

(73) Assignee: Archimed LLP, Sharnbrook, Bedford (GB)

(21) Appl. No.: 14/003,522

(22) PCT Filed: Mar. 6, 2012
(86) PCT No.: PCT/GB2012/050491

§ 371 Date: Sep. 6, 2013

(30) Foreign Application Priority Data
Mar. 7, 2011 (GB) 1103827.0
Mar. 16, 2011 (GB) 1104412.0
Jun. 17, 2011 (GB) 1110293.6

(51) Int. Cl. C12Q 1/26

(52) U.S. Cl. CPC C12Q 1/26
USPC 435/288.7

(57) ABSTRACT

A biosensor for detecting the concentration of an analyte in wound fluid, the biosensor being at least substantially free of catalase and comprising a sensing means comprising an oxidase enzyme and a hydrogen peroxide indicator means, the arrangement being such that, when a sample of wound fluid comprising the analyte is brought into contact with the sensing means, the oxidase enzyme reacts with the analyte to produce hydrogen peroxide which triggers the indicator means to indicate the presence of the analyte in the wound fluid.

### TECHNICAL FIELD

The invention relates to a biosensor, particularly for detection of metabolites that are oxidisable by a specific oxidase to yield hydrogen peroxide as a product, including lactate, urate and/or glucose.

### BACKGROUND TO THE INVENTION

With the emergence of interest in diagnostic tests for inflammatory diseases and infections, there is a growing need for the measurement of metabolites that accumulate as a result of inflammatory processes and/or infection. Actic acid (also referred to as lactate) is one such metabolite, being produced by both bacteria and leukocytes. It can be detected in various body fluids, due to its production in several important physiological processes, especially glycolytic respiration, occurring as a consequence of inflammation and infection. Urate is another metabolite produced as a terminal purine catabolite from precursors such as adenosine. Existing actate measurement products are mainly focussed on sports medicine, where lactate accumulation in the blood is recognised as a marker of exercised-induced glycolysis relevant to fitness and effectiveness of training exercises. Other diagnostic applications have proved elusive, due to the complexity of the biochemical pathways and cell physiology processes that contribute to its production.

Recent developments, however, have shown that localised lactate production in wounds reflects processes and conditions that affect wound healing. High levels of lactate (e.g. above 18 mM) are known to be deleterious to wound healing, while levels around 3 mM can be helpful. There is, therefore, an emerging need for simple, robust and stable detection systems for lactate in wound fluid to diagnose the healing status or predict the potential to heal.

Several lactate assay systems are already available and these have been used to advance clinical and scientific research into the role and significance of lactate. In sports medicine and fitness training lactate measurements are steadily increasing in popularity as a means to maximise the effectiveness of training programmes. The various types of lactate tests are as follows.

Laboratory analyser instruments are capable of measuring lactate concentrations in various bodily fluids, utilising well known colorimetric chemical/enzymatic procedures. Laboratory based electrochemistry systems are also available, typified by the “YSI 2700 Select Biochemistry analyzer”, which is capable of detecting numerous analytes, including lactate (see http://www.ysilifesciences.com/index.php?page=ysi-2700-select-bioprocess-monitoring).

There are a number of products for use outside of the laboratory, based on electrochemical or colorimetric techniques. These are either based on simple hand-held analytical instruments designed for use with blood samples (see http://www.lactate.com/) or on simple dip-sticks that change colour in the presence of lactate, and in which the colour is read by the hand-held instrument.

Although not yet used widely in clinical diagnostics, simple colour-change test strips are also known and have been described in the literature. For example, Shimojo et al, Clin Chem, 35, 9 1992-1994 (1989) described a method for making visually observable test-strips for detecting and determining lactate in whole blood. This system was based on strips of synthetic-fibre filter paper impregnated with the enzymes lactate oxidase (LOx) and horse radish peroxidase (HRP), together with the chromogenic chemicals 4-aminoantipyrene and N-ethyl-N-(2-hydroxy-3-sulfopropyl)-m-toluidene. These substances were applied as solutions and then dried to create a coated layer. The coated layer was adhered to a plain filter paper sample-application layer on one side and onto a thin plastic strip on the other side. When blood was applied to the plain filter paper layer, the cell-free blood components diffused through to the enzymes and chromogen layer. This incoming blood plasma re-dissolved the reagents, allowing them to react with any lactate present, resulting in generation of a colour, the density of which was directly proportional to lactate concentration. The colour was observed through a hole in the thin plastic carrier strip, on the opposite side to the filter paper that had trapped the red blood cells.

Despite the advances made with the existing methods of lactate detection and analysis, there is a need for several improvements as follows:
1) Test strips that can be brought directly into contact with the surface of a wound, so that test fluid can directly enter the diagnostic device. The existing test systems suffer from either a sample-receiving port that can't cope with viscous wound fluid samples, or the ingredients needed for disclosing the presence of lactate are not biocompatible, or the test system cannot be sterilised without losing activity.
2) There is always a loss of efficiency (precision, reliability, manufacturability etc.) caused by the drying process, yet the assay system components are not stable in a hydrated form.
3) Assays that are to be built into dressings (to create “smart dressings” that indicate the state of healing to an observer) need to be able to act over a relatively long, deferred time interval, rather than immediately on application to the wound.
4) The enzymatic reactions that generate colour from the lactate need to be protected from catalase that is always in the sample, in variable concentrations.
5) Semi-quantitative indications of concentration are needed to help the user gain the maximum benefit from the test.

The same general arguments and needs for new technology are applicable to urate test in human body fluids, although the relevance of urate testing to sports medicine has not yet been established. However, its relevance as a means of determining inflammation status is becoming recognized (Fernandez, M. L., Upton, Z., Edwards, H., Finlayson, K., and Shooter, G. K. (2011), Elevated uric acid correlates with wound severity. International Wound Journal.

Technology offering such improvements is needed.

### SUMMARY OF THE INVENTION

In a first aspect, the invention relates to a biosensor for detecting the concentration of an analyte in wound fluid, the biosensor being at least substantially free of catalase and comprising a sensing means comprising an oxidase enzyme and a hydrogen peroxide indicator means, the arrangement being such that, when a sample of wound fluid comprising the analyte is brought into contact with the sensing means, the oxidase enzyme reacts with the analyte to produce hydrogen peroxide which triggers the indicator means to indicate the presence of the analyte in the wound fluid

In a second aspect, the invention relates to a biosensor for detecting the concentration of an analyte in wound fluid, the biosensor comprising a first sensing means component comprising an oxidase enzyme in dried condition and a second indicator means component comprising a hydrogen peroxide indicator means, the arrangement being such that, when a sample of wound fluid comprising the analyte is brought into contact with the sensing means, the oxidase enzyme reacts with the analyte to produce hydrogen peroxide which triggers the indicator means to indicate the presence of the analyte in the wound fluid.

In a third aspect, the invention relates to means for a biosensor to detect the concentration of an analyte in wound fluid, the bio sensor comprising a sensing means comprising an oxidase enzyme in dried condition and being reversibly included, and a hydrogen peroxide indicator means, the arrangement being such that, when a sample of wound fluid comprising the analyte is brought into contact with the sensing means, the oxidase enzyme reacts with the analyte to produce hydrogen peroxide which triggers the indicator means to indicate the presence of the analyte in the wound fluid

The biosensor thus can provide a simple and easy-to-interpret visual signal of the presence of an analyte in the wound, providing an indication of its healing potential.

In use, wound fluid is brought into contact with the sensing means, and moisture activates the oxidase enzyme to carry out its sensing function.

By “reversibly included” is meant that the enzyme is not immobilised by covalent linkages to an insoluble polymer. It is not irreversibly cross-linked to an extent to which it prevents the enzyme from being diffusible.

The oxidase enzyme may be any suitable oxidase, according to the analyte the biosensor is designed to sense. Preferably the oxidase enzyme is lactate oxidase for sensing lactate levels in wound fluid or urate oxidase for sensing urate levels in wound fluid or glucose oxidase for sensing glucose levels in wound fluid. A combination of all three might be used if a composite integrated result is required, or a set of two or more individual devices might be contacted with a single wound to simultaneously measure the level of more than one marker (for example simultaneous measurements of lactate and urate in one wound to provide extra diagnostic information.

The sensing means and indicator means may thus form separate components of the biosensor, remaining distinct from each other prior to use of the biosensor. Alternatively, the sensing means and indicator means may be merged together as one component of the dressing, with the one component providing both the sensing means and indicator means functions. In one particularly preferred embodiment, the sensing means and indicator means are provided in a dry film as a single component. The dry film can be water soluble, e.g. comprising polyvinylacetate. Furthermore such water-soluble polymers can help to provide the indicator means.

One major advantage of providing the sensing means and indicator means together in a polymer film is that it may be fabricated together as one unit, for ease of application as a biosensor either alone or with other components.

As it is important to exclude catalase from the biosensor, the biosensor further preferably comprises a means for preventing the ingress of any molecule having a molecular weight greater than 200,000 into the sensing means, thereby allowing lactate to enter the biosensor from the wound, being oxidised to form hydrogen peroxide by the action of the lactate oxidase, the hydrogen peroxide thereby triggering the indicator means to indicate the presence of lactate in the wound.

The means for preventing the ingress of molecules larger than lactate into the sensing means is essential to prevent the ingress of materials such as catalase, which are present in wounds and can cause erroneous indications in the biosensor.

The means for preventing the ingress of any molecule having a molecular weight greater than 200,000 may take a variety of forms, for example a semi-permeable membrane allowing free passage of water and low molecular weight solutes, but preventing passage of high molecular weight solutes such as enzymes. Preferably the means for preventing the ingress of large molecules prevents the ingress of any molecule having a molecular weight greater than 100,000, more preferably greater than 50,000, most preferably greater than 25,000.

The biosensor can be sealed in packaging and comprising a sealed opening which, in use, is exposed and wound fluid is introduced to it. When sealed in packaging the sealed opening is the one and only opening in the packaging. In this embodiment, the biosensor is designed to remain in its packaging during use.

If sealed in packaging then the indicator means is visible through the packaging,

Although typically in dried condition, in some embodiments the sensing means and/or indicator means can be in moist condition and comprise water. In such a case, as suitable form is that of a hydrated hydrogel. Such a hydrogel is a material that has a solid network of polymeric material, extending over a length scale large in comparison to the molecular length scale. Such a network comprises liquid water as swelling agent and the solid-like nature of the network allows it to swell as more water is taken up.

Suitable hydrogels are disclosed in WO 03/090800. The hydrogel conveniently comprises hydrophilic polymer material. Suitable hydrophilic polymer materials include polyacrylates and methacrylates, e.g. as supplied by First Water Ltd in the form of sheet hydrogels, including poly 2-acrylamido-2-methylpropane sulphonic acid (polyAMPS) or salts thereof (e.g. as described in WO 01/96422), polysaccharides e.g. polysaccharide gums particularly xanthan gum (e.g. available under the Trade Mark Keltrol), various sugars, polycarboxylic acids (e.g. available under the Trade Mark Gantrez AN-169 BF from ISP Europe), poly(methyl vinyl ether co-maleic anhydride) (e.g. available under the Trade Mark Gantrez AN 139, having a molecular weight in the range 20,000 to 40,000), polyvinyl pyrrolidone (e.g. in the form of commercially available grades known as PVP K-30 and PVP K-90), polyethylene oxide (e.g. available under the Trade Mark Polyox WSR-301), polyvinyl alcohol (e.g. available under the Trade Mark Elvanol), cross-linked polyacrylic polymer (e.g. available under the Trade Mark Carbopol EZ-1), celluloses and modified celluloses including hydroxypropyl cellulose (e.g. available under the Trade Mark Klucel EEF), sodium carboxymethyl cellulose (e.g. available under the Trade Mark Cellulose Gum 7LF) and hydroxyethyl cellulose (e.g. available under the Trade Mark Natrosol 250 LR).

Mixtures of hydrophilic polymer materials may be used in a hydrogel.

In a hydrogel of hydrophilic polymer material, the hydrophilic polymer material is desirably present at a concentration of at least 1%, preferably at least 2%, preferably at least 5%, preferably at least 10%, more preferably at least 20%, yet more preferably at least 25%, or at least 30%, desirably at least 40% by weight based on the total weight of the gel.

A preferred hydrogel comprises poly 2-acrylamido-2-methylpropane sulphonic acid (poly AMPS) or salts thereof, preferably in an amount of about 20% by weight of the total weight of the gel.

In one embodiment, the oxidase enzyme, e.g. lactate oxidase, and the hydrogen peroxide indicator means are preferably dissolved in the water forming the swelling agent for the hydrogel. In another embodiment the oxidase enzyme is preferably dissolved in the water forming the swelling agent for the hydrogel and the hydrogen peroxide indicator means is adjacent and in contact with the hydrogel. For example the indicator means may comprise absorbent paper or any other suitable carrier such as another gel or an open porous foam. Typically the hydrogel is nearer the sealed opening than is the indicator means, so that any migration of material from the wound must first pass into the hydrogel before reaching the indicator means.

The hydrogen peroxide indicator is capable of providing a visually perceptible indicator that hydrogen peroxide has been formed in the sensing means.

Typically the hydrogen peroxide indicator means comprises a chromogenic material. The hydrogen peroxide formed oxidises the chromogenic material to provide a coloured indicator of the presence of hydrogen peroxide.

In a preferred embodiment the chromogenic material comprises iodide. This can be readily oxidised by the formed hydrogen peroxide to form iodine. In the presence of a complexing agent, such as starch or polyvinyl acetate, a wide range of bright colours can be generated to provide the visually perceptible indicator.

Optionally, the hydrogen peroxide indicator means comprises peroxidase enzyme. This can assist with the oxidation of the chromogenic material.

Typically the hydrogen peroxide indicator means provides a visual indicator which is in proportion to the concentration of hydrogen peroxide (and thus the detected species e.g. lactate) in the sensing means. This can be achieved in a number of ways, for example providing a plurality of indicator regions in the sensing means, each one adapted to be responsive to a different concentration of hydrogen peroxide. For example each region could have a differing amount of oxidase, peroxidase, chromogen or other reaction conditions. When the sensing means comprises a hydrated hydrogel, each indicator region could also comprises a differing degree of cross-linking, thus restricting the amount of lactate diffusing into each indicator region.

In one embodiment, the sensing means comprises oxidoreductase enzyme for the detection of glucose as the detected species.

In a further preferred embodiment, the sensing means can comprise lactate oxidase or urate oxidase and also comprise a control region which provides a visual indication of the presence of glucose. This control region will not contain any lactate or urate oxidase enzyme and instead will contain glucose oxidase enzyme. Typically the biosensor will contain a supply of pre-dosed glucose, although this can be provided by the wound exudate. When pre-dosed this gives the advantage that, upon use, it will be certain that some glucose is present, which is oxidised to yield hydrogen peroxide as pme pf tje [rpdicts by the glucose oxidase enzyme. This acts, in the same way as the rest of the sensing means, to provide a visual indicator that the biosensor is working.

Thus, the hydrogen peroxide indicator means can include a glucose-sensitive control region which is independent of the present of lactate or urate.

Other embodiments may be conceivable wherein the sensing means is responsive to is both lactate and glucose. For example the biosensor could be arranged to indicate detected quantities of both detected species, the indicator means giving a visual indication of levels of both species.

In another preferred embodiment, when the sensing means comprises a hydrated hydrogel, the hydrogel can also function as the means for preventing ingress of large molecules. This can be achieved, for example, by control over the degree of cross-linking.

Typically the biosensor is sterile, which can be achieved by exposing the sealed biosensor to sterilising radiation, such as gamma radiation.

In one embodiment, the biosensor can also include a diffusion means situated between the sealed opening and the sensing means. This can be desirable if the speed of response to the presence of the analyte in the wound is too rapid. The diffusion means can thereby act to slow down the transmission of wound fluid to the sensing means. The diffusing means may comprise a wide range of materials. The diffusing means may be dry or partially or fully saturated with water.

In one preferred embodiment, the biosensor is not intended for direct application onto a wound. Instead, wound fluid is sampled and brought into contact with the bio sensor, away from the wound.

In another embodiment the biosensor is intended to be placed directly onto a wound. In one embodiment the biosensor forms part of a larger composite dressing. For example it can be part of a foam dressing which requires ethyl oxide to sterilise it. The sterilised bio sensor can then remain protected from the harmful ethyl oxide sterilisation process by its packaging. Thus the composite dressing can be prepared in sterile form, the biosensor sterilised by radiation and the remainder being sterilised by ethyl acetate. When the dressing is desired to be used the removable seal is removed and the dressing applied in the usual manner.

The invention will now be illustrated with reference to the following figures, in which

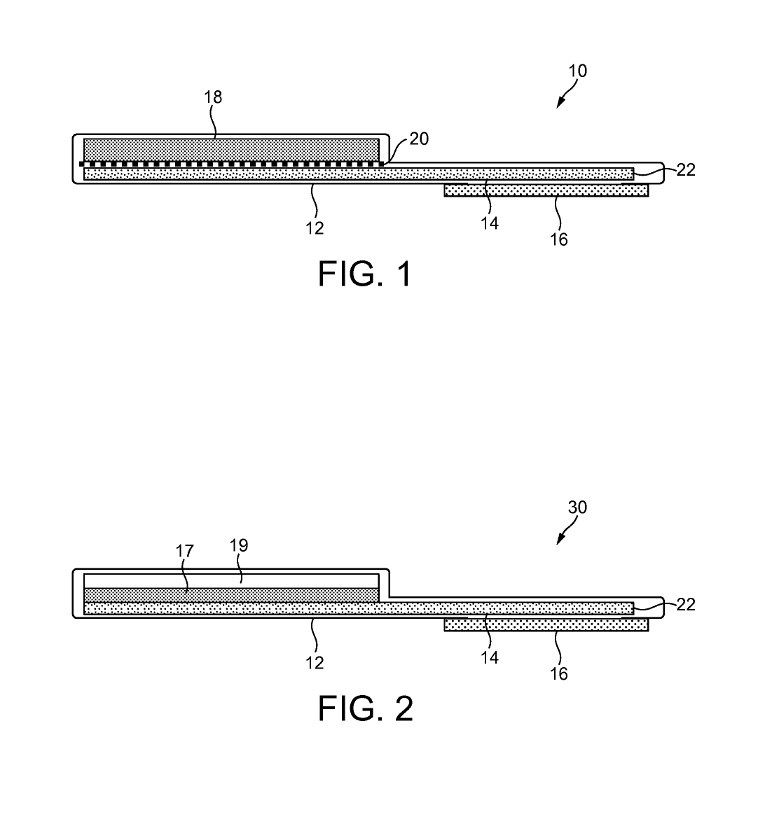

FIG. 1 is a schematic representation of a biosensor in accordance with the present invention.

FIG. 2 is a schematic representation of another biosensor in accordance with the present invention.

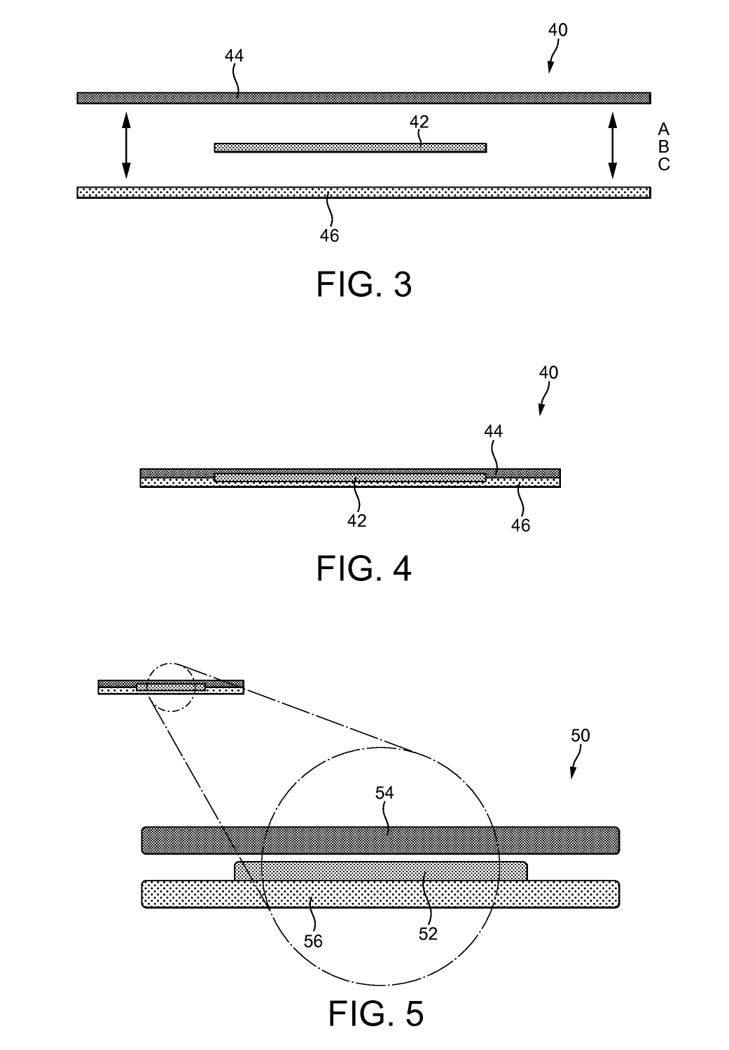

FIG. 3 is a schematic representation of another biosensor in accordance with the present invention.

FIG. 4 is a close up view of the biosensor shown in FIG. 3.

FIG. 5 is a schematic representation of another biosensor in accordance with the present invention.

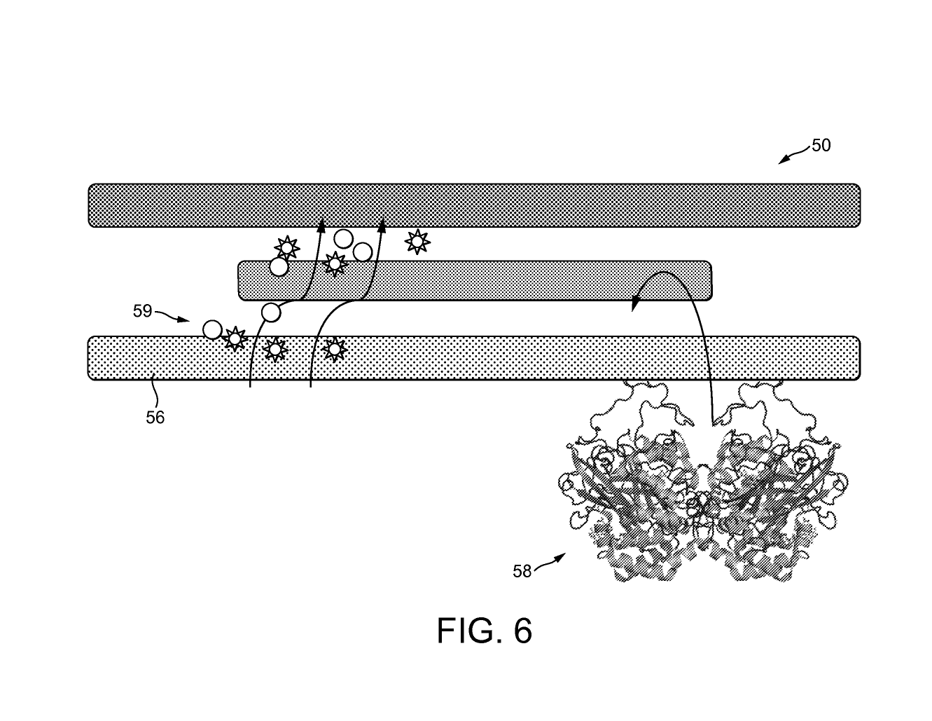

FIG. 6 is a schematic representation of another biosensor in accordance with the present invention.

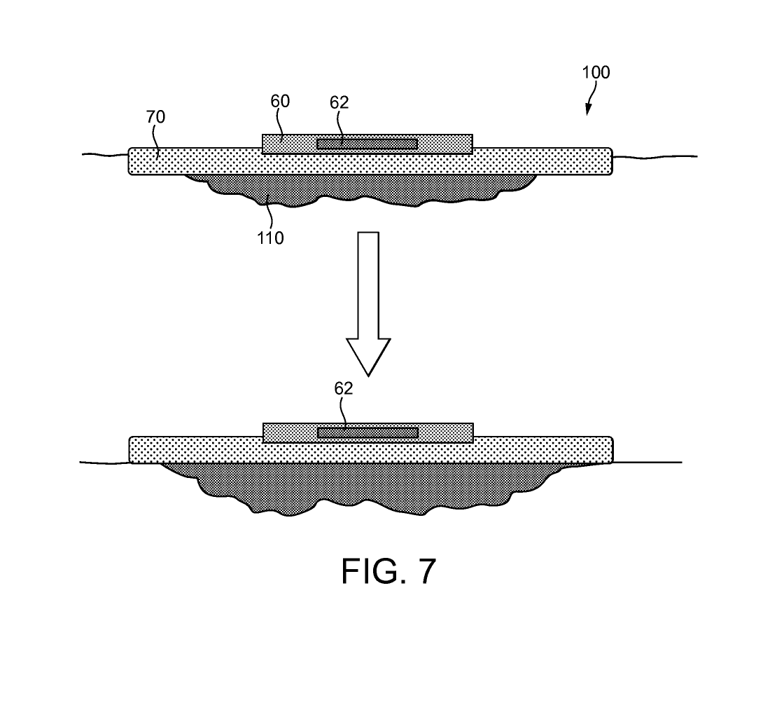

FIG. 7 shows a schematic representation of a biosensor in accordance with the present invention in use.

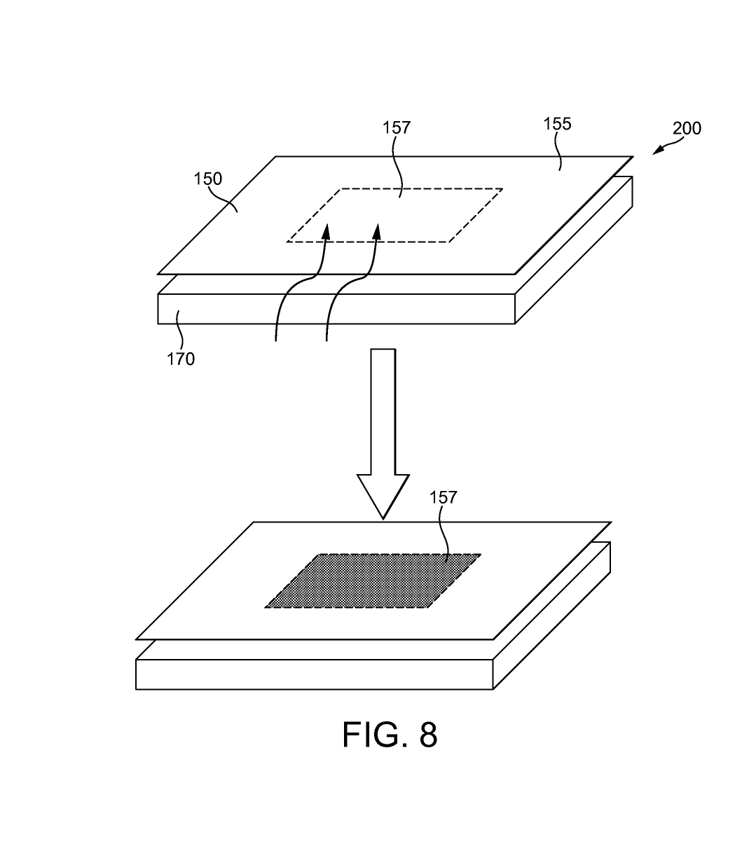

FIG. 8 shows a schematic representation of another bio sensor in accordance with the present invention in use.

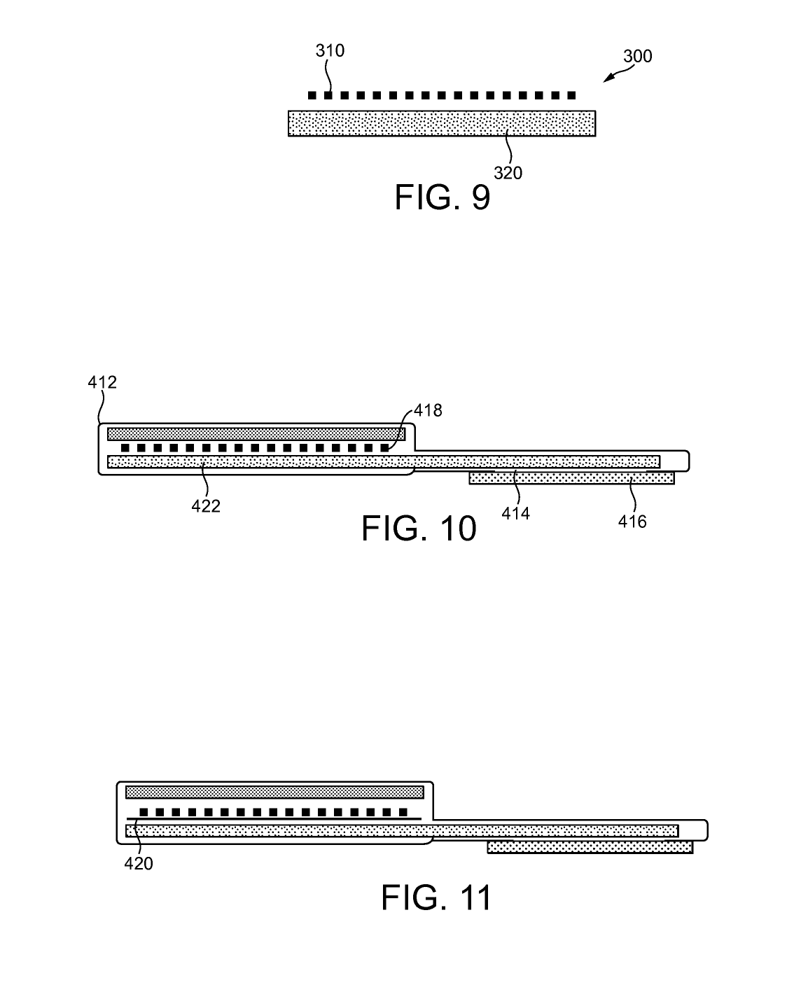

FIG. 9 is a schematic representation of another biosensor in accordance with the present invention.

FIG. 10 is a schematic representation of another biosensor in accordance with the present invention.

FIG. 11 is a schematic representation of another biosensor in accordance with the present invention.

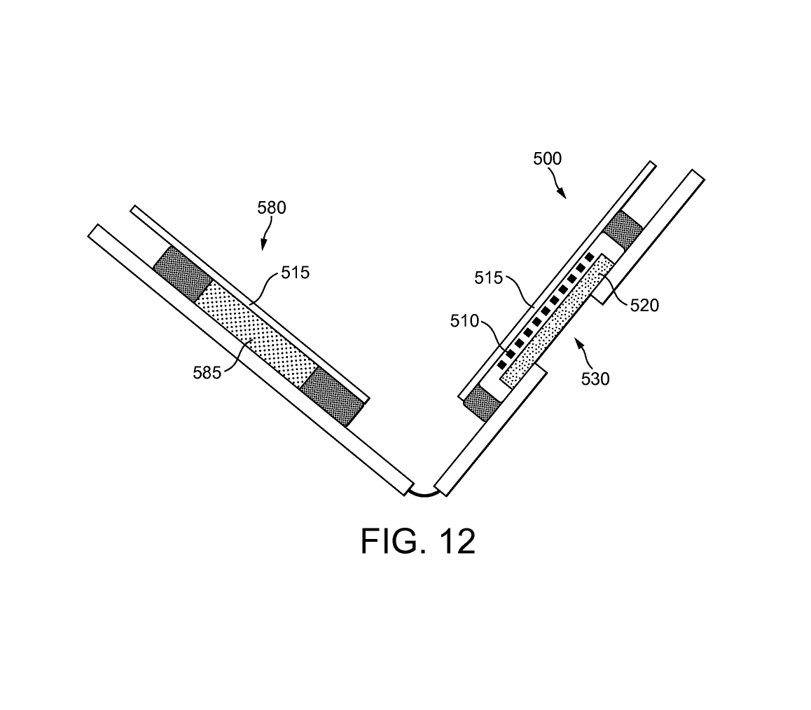

FIG. 12 is a schematic representation of another biosensor in accordance with the present invention.

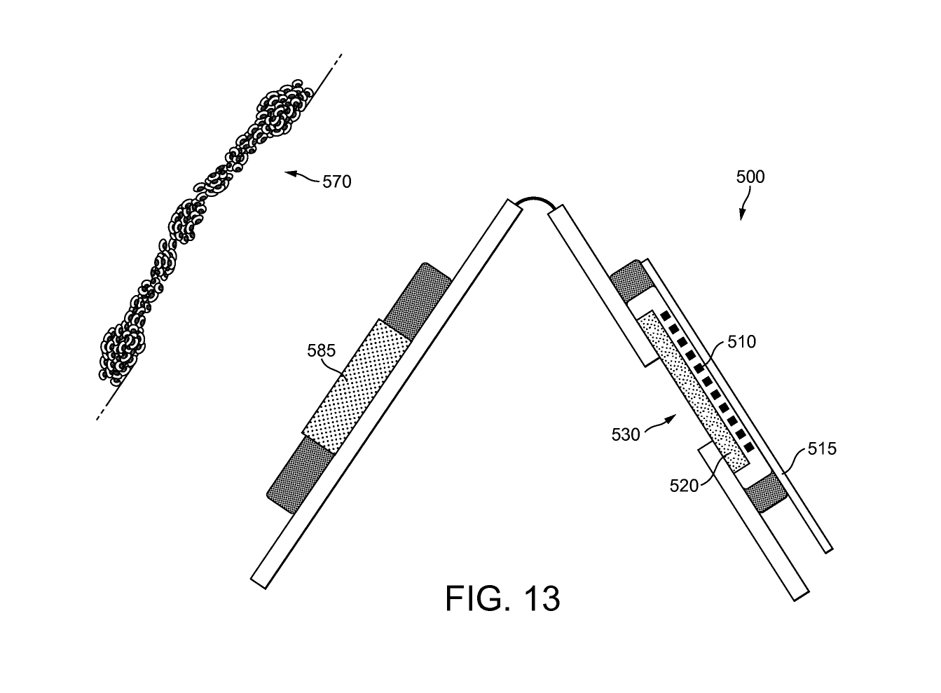

FIG. 13 is a schematic representation of the biosensor shown in FIG. 12.

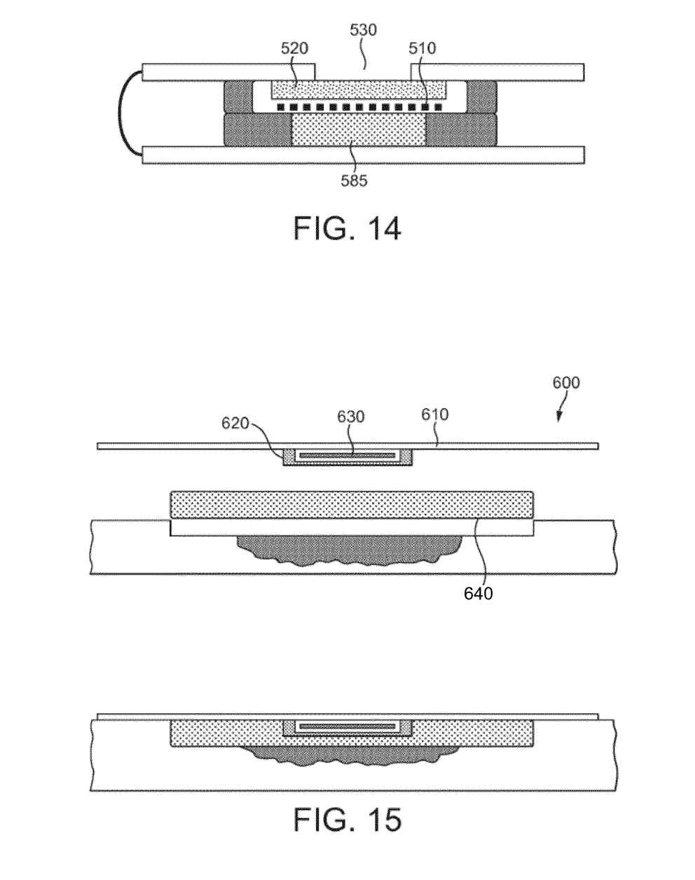

FIG. 14 is a schematic representation of the biosensor shown in FIG. 12 in use.

FIG. 15 is a schematic representation of another biosensor.

Turning to the figures, FIG. 1 shows a biosensor 10 sealed in clear transparent packaging 12, and having an opening 14 covered by removable seal 16.

The biosensor 10 comprises a sensing means 18 which comprises a hydrated hydrogel containing lactate oxidase, horse radish peroxidase enzyme, iodide as the chromogen material, and starch, and is essentially free of catalase.

The bio sensor 10 also comprises semi-permeable membrane 20 which allows the free passage of water, lactate and other low molecular weight solutes but prevents passage of high molecular weight solutes such as enzymes e.g. catalase.

The biosensor 10 also comprises an absorbent wick material 22 which provides a fluid diffusion path from the opening 14 to the sensing means 18 and comprises a fabric saturated with water, although many other versions are possible.

In use the seal 16 is removed and the opening 14 is placed over a wound in the skin of a human or animal subject.

Wound exudates then diffuses into the biosensor through opening 14 and diffuses along the absorbent wick 22. Once at the semi-permeable membrane 20 only the lactate and other low molecular weight solutes continue to diffuse into the hydrogel 18.

Once in the hydrogel 18 the lactate oxidase causes oxidation of the lactate to form hydrogen peroxide. The formed hydrogen peroxide then oxidises the iodide with the action of the peroxidase enzyme to form iodine. The iodine then complexes with the starch which forms a distinctive blue colour. This causes a visual indication in a change of colour of the hydrogel, which is visible through the clear transparent packaging.

FIG. 2 shows a similar arrangement to that shown in FIG. 1, showing a bio sensor 30 sealed in a clear transparent packaging 12 and having an opening 14 covered by removable seal 16.

The biosensor 30 comprises a sensing means comprising a hydrated hydrogel 17 containing lactate oxidase. The hydrogel is cross-linked to such an extent that it allows the free passage of water, lactate and other low molecular weight solutes but prevents the passage of high molecular weight solutes such as enzymes.

The sensing means also comprises a hydrogen peroxide indicator means 19 comprising iodide and starch dried into absorbent paper.

The bio sensor 30 also comprises an absorbent wick 22 which provides a fluid diffusion path from the opening 14 to the sensing means 18 and comprises a fabric saturated with water, although many other versions are possible.

In use the seal 16 is removed and the opening 14 is placed over a wound in the skin of a human or animal subject.

Wound exudates then diffuses into the biosensor through opening 14 and diffuses along the absorbent wick 22. Once at the hydrogel 17 only the lactate and other low molecular weight solutes continue to diffuse into the hydrogel 17.

Once in the hydrogel 17 the lactate oxidase causes oxidation of the lactate to form hydrogen peroxide. The formed hydrogen peroxide then diffuses to the indicator means 19 and reacts with the iodide to form iodine. The iodine then complexes with the starch which forms a distinctive blue colour. This causes a visual indication in a change of colour of the indicator means 19, which is visible through the clear transparent packaging.

FIG. 3 shows a biosensor 40 comprising single component 42 comprising both the sensing means and indicator means functions, and is essentially free of catalase. The biosensor also has a cover film 44 and a molecular weight selective barrier 46 to prevent the ingress of catalase.

FIG. 4 shows the biosensor of FIG. 3 in assembled form.

FIG. 5 shows another biosensor 50 comprising a dry single component 52 providing both the sensing and indicator functions. Also provided is an adhesive cover film 54 and a molecular weight selective barrier 56 to prevent the ingress of catalase.

FIG. 6 shows the biosensor 50 of FIG. 5 in use. Shown is a large molecule such as catalase 58 being rejected by the molecular weight selective barrier 56. However smaller molecules 59, are allowed into the sensing means.

FIG. 7 shows a composite wound dressing 100 sitting on top of the surface of a wound 110. The wound dressing 100 comprises a biosensor 60 adhered to a wound dressing component 70, such as a foam pad. The biosensor contains indicator means 62.

After some time, wound fluid passes through the wound dressing component 70 and enters the biosensor 60, carrying with it some concentration of the analyte, e.g. lactate. Hydrogen peroxide is produced and the indicator means 62 changes colour and provides a visible indication.

FIG. 8 shows a composite wound dressing 200 comprising a biosensor 150 adhered to a wound dressing component 170, such as a foam pad. The biosensor comprises a sensing means component 155 and an indicator means component 157.

After some time, wound fluid passes through the wound dressing component 170 and enters the biosensor 150, carrying with it some concentration of the analyte, e.g. lactate. Hydrogen peroxide is produced and the indicator means 157 changes colour and provides a visible indication.

FIG. 9 shows another biosensor 300, comprising a self-indicating dry film 310 which changes colour in the presence of hydrogen peroxide and contains stabilised oxidase enzyme. Also shown is an optional backing pad or sheet 320 which could be a hydrogel in aqueous or dry condition.

FIG. 10 shows a biosensor 400 sealed in clear transparent packaging 412, and having an opening 414 covered by removable seal 416.

The biosensor 400 comprises a sensing means 418 which is a dry film containing stabilised oxidase enzyme, horse radish peroxidase enzyme, iodide as the chromogen material, and starch, and is essentially free of catalase.

The bio sensor 400 also comprises an absorbent wick material 422 which provides a fluid diffusion path from the opening 414 to the sensing means 418 and comprises a fabric saturated with water, although many other versions are possible.

FIG. 11 shows a variant of the biosensor shown in FIG. 10, which includes a molecular weight selective membrane 420 which allows the free passage of water, lactate and other low molecular weight solutes but prevents passage of high molecular weight solutes such as enzymes e.g. catalase.

FIG. 12 shows a biosensor 500, hingedly attached to a sampling pad 580. The biosensor comprises a self-indicating dry film 510 which changes colour in the presence of hydrogen peroxide and contains stabilised oxidase enzyme. Also shown is an optional backing pad or sheet 520 which could be a hydrogel in aqueous or dry condition. The biosensor also comprises a viewing window 530.

The sampling pad 580 comprises a foam sampling pad 585. Also provided are peel-off covers 515.

FIG. 13 shows the biosensor of FIG. 12 in use, wherein the peel-off cover 515 for the sampling pad is removed and the sampling pad 585 is brought into contact with a wound 570.

Once sampled, FIG. 14 shows how the peel-off cover for the biosensor is removed and the sampling pad 585 brought into contact with dry film 510.

The test results can then be seen through transparent window 530.

FIG. 15 shows a biosensor 600 comprising a transparent adhesive cover film 610 and a semi-permeable membrane 610 adhering around its edges to create an enclosed space. The biosensor comprises a dry film sensor element 630 behind the semi-permeable membrane 610 with lactose oxidase reversibly contained within the film and all other ingredients required to achieve an indicator reaction in the presence of lactate. Also shown is an absorbent foam wound dressing 620 which has been applied to a wound.

The bio sensor 600 can then be places on top of the dressing 620, and provide an indication of the level of lactate in the wound in use.

1. A biosensor for detecting the concentration of an analyte in wound fluid, the biosensor comprising:
a sensing means comprising an oxidase enzyme; and
a hydrogen peroxide indicator means, wherein when a sample of wound fluid comprising the analyte is brought into contact with the sensing means, the oxidase enzyme reacts with the analyte to produce hydrogen peroxide, which triggers the indicator means to indicate the presence of the analyte in the wound fluid.

2. A biosensor according to claim 1, further comprising a first component comprising the sensing means with the oxidase enzyme in dried condition and a second component comprising the hydrogen peroxide indicator means.

3. A biosensor according to claim 1, wherein the oxidase enzyme is reversibly included.

4. (canceled)

5. A biosensor according to claim 1, wherein the biosensor is at least substantially free of catalase.

6. A biosensor according to claim 5, wherein the oxidase enzyme is reversibly included.

7-9. (canceled)

10. A biosensor according to claim 1, wherein the sensing means comprises a hydrated hydrogel.

11. A biosensor according to claim 1, wherein the oxidase enzyme comprises lactate oxidase, urate oxidase, glucose oxidase or mixtures thereof.

12. A biosensor according to claim 1, wherein the hydrogen peroxide indicator means comprises a chromogenic material.

13. A biosensor according to claim 12, wherein the chromogenic material comprises iodide.

14. A biosensor according to claim 1, wherein the sensing means and the indicator means are provided together in one film component, particularly a polymeric film component.

15. A biosensor according to claim 1, wherein the biosensor further comprises a peroxidase enzyme.

16. A biosensor according to claim 1, wherein the hydrogen peroxide indicator means provides a visual indicator which is in proportion to the concentration of hydrogen peroxide in the sensing means.

17. A biosensor according to claim 1, further comprising a means for preventing the ingress of any molecule having a molecular weight greater than 200,000 into the sensing means.

18. A biosensor according to claim 17, wherein the means for preventing the ingress of molecules larger than lactate into the sensing means comprises a semi-permeable membrane.

19. A biosensor according to claim 17, wherein the sensing means comprises a hydrated hydrogel and the means for preventing the ingress of molecules larger than lactate into the sensing means is provided by the hydrogel.

20. A biosensor according to claim 1, wherein the biosensor is sterile.

21. A composite dressing comprising a biosensor according to claim 1.

22. A biosensor according to claim 1, wherein the indicator means comprises a hydrated hydrogel.

23. A biosensor according to claim 1, wherein the sensing means and the indicator means are provided together in a polymeric film component.
